(19) United States
(12) Patent Application Publication
(10) Pub. No.: US 2008/0234592 A1
(43) Pub. Date: Sep. 25, 2008

(54) Method and apparatus for generating an electrocardiogram

(76) Inventors: Meng Huat Damian Lim, Singapore (SG); Agustin Marjanti, Singapore (SG); Chin Han Kok, Singapore (SG); May Hui Shirley Koh, Singapore (SG); Xin Liu, Singapore (SG); Kok Beng Neo, Singapore (SG)

Correspondence Address: JACOBSON HOLMAN PLLC, 400 SEVENTH STREET N.W., SUITE 600, WASHINGTON, DC, 20004, US

(21) Appl. No.: 11/905,385

(22) Filed: Sep. 28, 2007

(30) Foreign Application Priority Data
Sep. 23, 2006 (SG) 200606813-4

(51) Int. Cl. A61B 5/0402

(52) U.S. Cl. 600/509

(57) ABSTRACT

A method of recording information for use in generating an electrocardiogram indicative of a health status of a user's heart, the method being operable using a device (1) which includes:
a housing (2);
a processor (3) disposed in the housing (3);
first and a second input terminal (4a,4b) disposed on the housing (2), wherein the first and second input terminals (4a,4b) are operatively connected to the processor (3); and
a display (5) operatively connected to the processor (3);
wherein the method includes the steps of:
   (i) contacting a right and left thumb (8a,8b) of the user with the first and second input terminals (4a,4b) respectively; and
   (ii) the processor (3) detecting an ECG signal formed between the first and second input terminals (4a,4b), wherein the ECG signal is indicative of the health status of the user's heart.

### TECHNICAL FIELD

The present invention relates to a method and apparatus for generating an electrocardiogram of particular but by no means exclusive application in the detection of persons displaying symptoms of heart diseases.

### BACKGROUND OF THE INVENTION

One existing method of screening people for heart diseases involves the use of a clinical 12-lead electrocardiograph to measure the electrical activities of the heart. For example, any person with a measured ECG that exhibits an abnormal characteristics pattern may be considered to be potentially suffering from a heart disease or disorder of the circulatory system. This may also be a key indicator suggesting a possible life threatening heart disease that could result in heart attack and/or cardiac arrest. However, clinical 12-lead ECGs can be relatively complicated to perform and are handled and interpreted only by trained medical professionals. Most individuals will have to visit the clinic or hospital to have their ECG measured and interpreted. This can be particularly inconvenient for elderly and disabled people who may lack the mobility to attend the hospital or clinic.

Lead-1 type ECG devices are relatively simpler to use than 12-lead ECG devices, however, they tend to be designed only for usage by the patients suffering from heart arrhythmias. Lead-1 type ECG devices normally require users to place the device directly on the chest in contact with the skin. The ECG that is recorded is then transmitted through an acoustic means via the phone network to a processing server where the results of the ECG may be interpreted. These types of prior art devices merely serve as “event recorders”—that is, they generally do not provide any immediate indication to the user as to whether the user is suffering from a heart abnormality.

Prior art devices of the type described above exhibit a host of other problems including the fact that they are less convenient for users to record an in public places—particularly for female users. Existing prior art devices also tend to record and store only a limited number of ECG readings at a time which may not be useful for detection of any abnormalities. Another problem associated with the prior art is that they are unable to ascertain the quality of the recorded ECG (eg. baseline of the ECG might wander, or, the ECG signal may be disrupted by other noises) since there is no LCD for the user to view the recorded ECG.

### SUMMARY OF THE INVENTION

The present invention seeks to alleviate at least one of the problems described above in relation to the prior art.

The present invention may involve several different broad forms. Embodiments of the invention may include one or any combination of the different broad forms herein described.

The present invention provides a method of recording information for use in generating a Lead-1 type electrocardiogram indicative of a health status of a user's heart, the method being operable using a device which includes:
a housing;
a processor disposed in the housing;
a first and a second input terminal disposed on the housing, wherein the first and second input terminals are operatively connected to the processor; and
a display operatively connected to the processor;
wherein the method includes the steps of:
   (i) contacting a right and left thumb of the user with the first and second input terminals respectively; and
   (ii) the processor detecting an ECG signal formed between the first and second input terminals, wherein the ECG signal is indicative of the health status of the user's heart.

In a second broad form, the present invention provides a method of recording information for use in generating a Lead-2 type electrocardiogram indicative of a health status of a user's heart, the method being operable using a device which includes:
a housing;
a processor disposed in the housing;
a first and a second input terminal disposed on the housing, wherein the first and second input terminals are operatively connected with the processor; and
a display operatively connected to the processor;
wherein the method includes the steps of:
(i) contacting a user's right thumb with the first input terminal, and, contacting a region of skin on the user's body with the second input terminal; and
(ii) the processor detecting an ECG signal formed between the first and second input terminals, wherein the ECG signal is indicative of an operating status of the user's heart.

Preferably, the detected ECG signal is also recorded by the present invention.

Preferably, the present invention also includes the step of outputting an ECG to the display by reference to the recorded ECG signal between the first and second input terminals. The ECG may be displayed as a graph of voltage versus time, or alternatively, in text format which indicates information about specific characteristics of the signal—for instance the ST interval, QRS complex and the like.

Typically, the housing includes a rectangular-shaped configuration having an upper and lower surface, and, left and right ends. The left and right ends of the housing may be defined by reference to a point of view of a user when ordinarily reading the display.

Typically, the display includes a liquid crystal display (LCD). However, it would be appreciated by a person skilled in the art than any number of other suitable display means may be used in place of the LCD. For instance, an LED display may be used instead. Preferably, the display may be disposed on the upper surface of the housing.

Also preferably, the first and second input terminals include electrically conductive panels made for instance from a metal. The first and second input terminals may also be mounted on the upper surface of the housing. More preferably, the first input terminal may be disposed on the right end of the housing whilst the second input terminal may be disposed on the left end of the housing. The first and second input terminals also cover side edges of the ends of the housing. Typically, the second input terminal may also include a lead extending away from the housing. Preferably, the lead may be selectively extendable and retractable relative to the housing. Advantageously, the extendable lead may be particularly useful for a user who is taking a Lead-2 type ECG reading.

Preferably, the device also includes a memory store such as flash memory, eSRAM, RAM or the like which is operably connected to the processor. Preferably, the present invention includes a step of recording the detected ECG signal in the memory store. Preferably, the memory store may be adapted to store at least about 20 recordings of ECG signals of the user's heart. Advantageously, by being able to record a number of ECG signals in this manner, a historical record of the user's heart function may be maintained for later analysis by a medical practitioner.

In certain embodiments, the present invention may be integrated into a portable computing device including at least one of a mobile phone, a notebook computer, and/or a PDA.

Typically, the present invention includes a communication interface including at least one of a USB-compatible connector and/or a wireless communication interface based on Bluetooth or Wi-Fi protocols so that it may communicate with external devices.

Typically, the step (i) of the first broad form of the present invention may involve contacting the first and second input terminals with the right and left thumbs of the user respectively for a period of about approximately 20 seconds in order to take a reading. Similarly, the step (i) of the second broad form of the present invention may involve contacting the first and second input terminals with the user's right thumb, and, a region of skin on the user's body respectively, for a period of about approximately 20 seconds in order to take a reading.

Preferably, in step (ii) of the second broad form of the present invention, the region of skin on the user's body surface against which the second input terminal is contacted with may typically include a region of skin disposed on a lower-left section of the user's abdomen.

Preferably the present invention may also include the processor measuring the user's heart rate by reference to the ECG signal and outputting a representation of the user's heart rate on the display. The present invention may be adapted to allow the user to either output the user's heart rate and ECG on the display concurrently, or, interchangeably.

Preferably, the present invention includes a step of reducing a common noise component associated with the detected ECG signal. Typically, this step may include the use of a noise-reduction system wherein the noise-reduction system may include a high-input-impedance instrumentation amplifier and/or a common mode operational amplifier. Typically, the noise reduction module derives a common-mode voltage signal from between the first and second input terminals during recording of the detected ECG signal, inverts the derived common-mode voltage signal, and drives the inverted common-mode voltage signal back into the user via a metal plate which is placed in contact with the user's skin during recordal of the ECG signal. Typically, the metal plate may be disposed on the lower surface of the device and may be operably connected to the processor of the device. Advantageously, the use of a noise reduction system may assist the device in recording a relatively clearer and more accurate ECG signal.

Preferably, the present invention may include the step of processing the recorded ECG signal in order to identify a characteristic of the ECG signal which may be indicative of an abnormality in the health status of the user's heart. More preferably, this step may involve comparing the characteristic of the detected ECG signal against a predetermined abnormality criteria in order to determine if an abnormality exists.

Typically, the heart abnormality may include an arrhythmia such as bradycardiac, tachycardiac, and premature ventricular contraction.

Typically, the characteristic of the detected ECG signal which may be compared against the predetermined abnormality criteria in order to detect an abnormality may include a segment of the ECG signal such as:
(a) an ST segment of the ECG signal;
(b) a PR segment of the ECG signal;
(c) a PR interval of the ECG signal;
(d) a QT interval of the ECG signal;
(e) a QRS complex of the ECG signal.

Typically, the predetermined abnormality criteria may include at least one parameter defining how the characteristic of the detected ECG signal should typically behave when the user's heart is functioning abnormally. For instance, the criteria may require that the ST segment be of a particular duration.

Typically, the present invention may also include the step of alerting the user that an abnormality exists if the characteristic of the detected ECG signal matches the predetermined abnormality criteria. For instance, a warning message may be output to the display. Alternatively, the present invention may include a light emitter device and/or an audio alarm which is programmed to alert the user to the detection of an abnormality. In certain embodiments, the present invention may also be adapted to transmit an emergency message to a local hospital, medical centre of the like when a recording of an ECG signal indicates that the user's heart function has deteriorated to a predetermined threshold level whereby it is imperative that immediate medical attention be sought. A warning of this nature may be transmitted via the Internet—for instance, if the device is in proximity to a wireless hotspot, the device may automatically connect to the wireless access point and send an email message containing details of the abnormality, and the location of the user (which may be determined by reference to the location of the wireless hotspot), to the nearest hospital or medical centre.

Typically, the step of comparing the characteristic of the detected ECG signal against the predetermined abnormality criteria may be performed by the processor of the device, or, an external server communicably connected to the device via a communication link. Advantageously, the use of an external server to process recorded ECG signal may be convenient in that it may provide additional processing power to complement a limited power capacity of the user's device. The external server may also provide additional memory space to store recordings made by the device.

Typically, the external server may also include a database for storing the recorded ECG signals sent to it from the user's device.

Typically, the communication link may include the Internet, an Intranet, a LAN a WAN or any other communication network considered to be suitable by a person skilled in the art. Typically, the device may be adapted to communicate directly across the communication link with the external server, or alternatively, it may be interfaced with an intermediary terminal which is adapted to communicate with the external server via the communication link. For instance, the recorded ECG signal may be uploaded into the intermediary terminal (such as a PC) which has a broadband or dial-up connection to the Internet whereby the intermediary terminal may be adapted to transmit the recorded ECG signal to the external server via the Internet for processing.

Typically, where the external server is used to process the detected ECG signal, the external server may initiate the transmission of a warning message to the user where an abnormality in the health status of the user's heart is detected. The external server may be adapted to transmit the warning message via at least one of the following messaging formats:
(a) e-mail;
(b) SMS;
(c) Facsimile;
(d) a telephone call.

Alternatively, the external server may communicate with the device via the communication link whereby if an abnormality is detected, the device may be caused to output an appropriate warning message on the display.

Typically, the warning message may include at least one of:
(a) information indicating the nature of the abnormality; and/or
(b) information about a treatment for the abnormality.

The present invention may also include the use of a Web site interface to allow a user to review his/her information relating to recordings of ECG signals that he/she has sent to the external server for processing. Any computing device such as a PC having a suitable Web browser and Internet access may allow the user to interact with the Web site. The Web site may provide information including, amongst other things:
(a) details of each recording such as the measured heart rate associated with the recording;
(b) details of various segments of each recorded ECG signal such as the ST interval, QRS complex and the like;
(c) details of the time at which each recording was made;
(d) details of any abnormality(s) that have been detected in a recording;
(e) details of any advice provided by a medical practitioner that has been requested by the user to review a recording;

Typically, the Web site may be hosted on a Web server which typically includes the external server itself. The Web site may include a password-access scheme in order to ensure that personal information of each user may be maintained a confidential manner. The Web site may also provide a user-interface by which the user is able to manage the uploading of a recording directly to the external server. For instance, if the recording of a particular ECG signal is stored on the device and the device is interfaced with the PC via a USB cable for instance, then the user may, via the Web site interface initiate a file transfer of the recording to the external server for processing as required.

The present invention may include the step of maintaining the device in continuous contact with the user's body in accordance with the method steps of either of the first and second broad forms of the present invention whereby the present invention may be adapted to continuously monitor the health status of the user's heart. In this regard, the ECG signal may be processed regularly by either the device itself or the external server in order to determine if an abnormality arises. This may be particularly convenient in circumstances where the user has a serious heart condition and it is necessary to closely monitor abnormalities and/or deterioration in its condition. For instance, if the device is wirelessly connected to the intermediary terminal, detected ECG signals may be regularly uploaded to the intermediary terminal in real-time and transmitted to the external server for processing via the communication link. In effect, the present invention may function as a heart monitoring device if so desired.

Preferably, the present invention may include a control means for selecting a mode of operation—that is, a manual monitoring mode, and, a continuous monitoring mode. For instance one mode may involve the present invention being used to detect and/or record ECG signals of the user's heart manually as required. Another mode may involve the present invention being adapted for continuous monitoring and/or recordal of the user's heart (i.e. when the present invention is kept in contact with the user's body.)

Typically, at least some of the method steps of the present invention may be implemented by an administrator. Typically, the administrator may include a person who manually performs the method step(s), or, may include a computerised system which may be programmed to perform the method step(s) automatically. Typically, the administrator includes a person working in combination with a computerised system. For instance, the administrator includes a hospital or medical practice.

Typically, a fee may be charged by the administrator in exchange for the user being able to upload recorded ECG signals to the external server for storage and/or processing. Typically a fee may also be charged in exchange for the external server sending a warning message to the user when a heart abnormality is detected as a result of a comparison being made between the ECG signal and a predetermined abnormality criteria.

Typically, a program may be stored in the memory store of the device, wherein the program is executable by the processor of the device to perform any one of the method steps of the present invention. Any software considered suitable by a person skilled in the art may be used.

In a third broad form, the present invention provides a device adapted for use in accordance with any one of the first and/or second broad forms of the present invention.

In a fourth broad form, the present invention provides a server adapted for use in accordance with any one of the first and/or second broad forms of the present invention.

In a fifth broad form, the present invention provides a computer readable medium for storing a computer program executable to perform the method steps in accordance with at least one of the first and/or second broad forms of the present invention described herein.

Typically, the present invention may be battery-powered or powered by a mains power supply.

Typically, in certain embodiments, the present invention includes a device for measuring the user's blood pressure.

### BRIEF DESCRIPTION OF THE DRAWINGS

The present invention will become more fully understood from the following detailed description of a preferred but non-limiting embodiment thereof, described in connection with the accompanying drawings, wherein:

FIG. 1 depicts a flow diagram of steps to be performed in accordance with a first embodiment of the present invention.

FIG. 2 depicts a top view of a device used in accordance with a first embodiment of the present invention wherein the upper surface of the housing is visible;

FIG. 3 depicts a rear view of the device in FIG. 2 wherein the lower surface of the housing is visible;

FIG. 4 depicts a magnified view of a display of the device in FIG. 2 showing an example display output including characteristics of the ECG, and the user's heart rate;

FIG. 5 depicts the device shown in FIG. 2 in use to detect an ECG signal in order to generate a Lead-1 type ECG;

FIG. 6 depicts the device shown in FIG. 2 in use to detect an ECG signal in order to generate a Lead-2 type ECG;

FIG. 7 depicts an electrical schematic diagram of the device used in accordance with the first embodiment;

FIG. 8 depicts a further embodiment of the present invention in which processing of the detected ECG signal of the user is processed by an external server.

### MODES FOR CARRYING OUT THE INVENTION

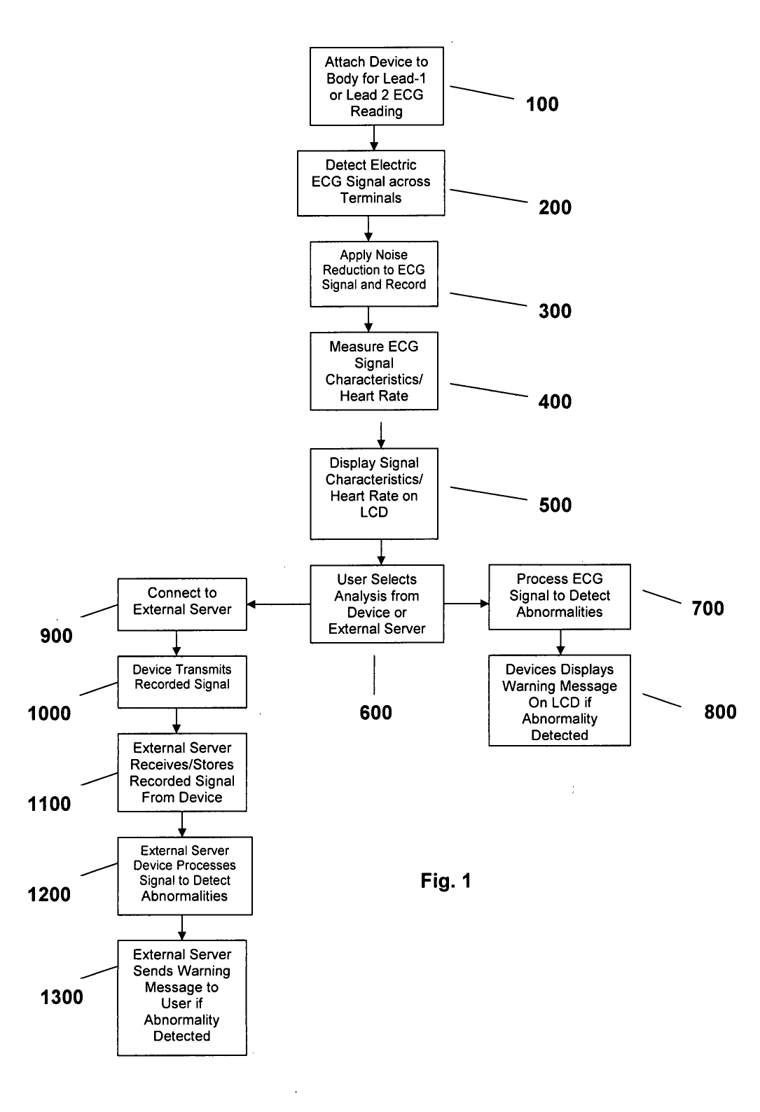

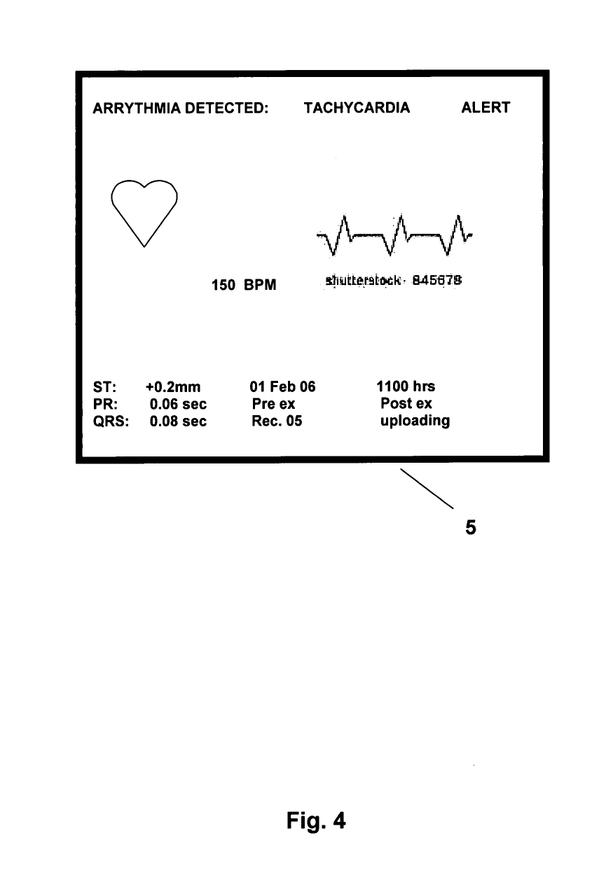

A first embodiment of the present invention will now be described by reference to FIGS. 1-8.

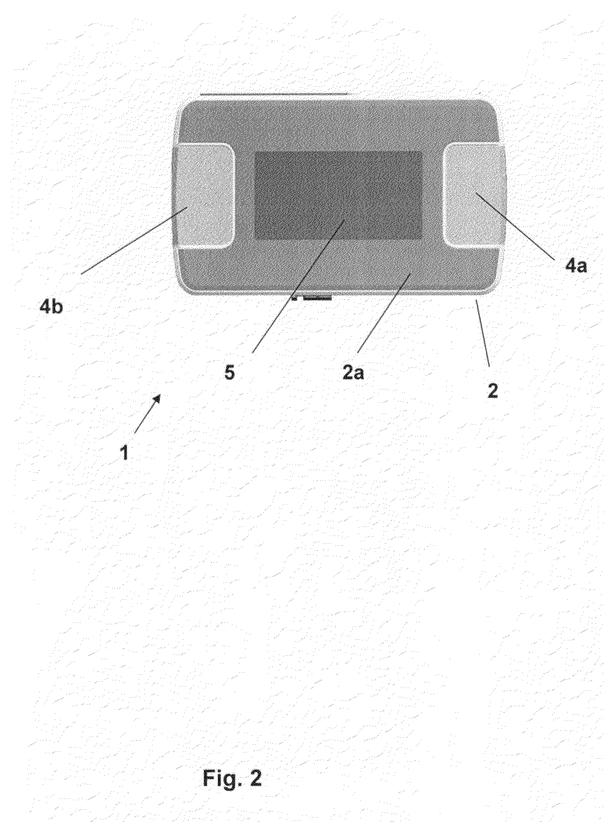

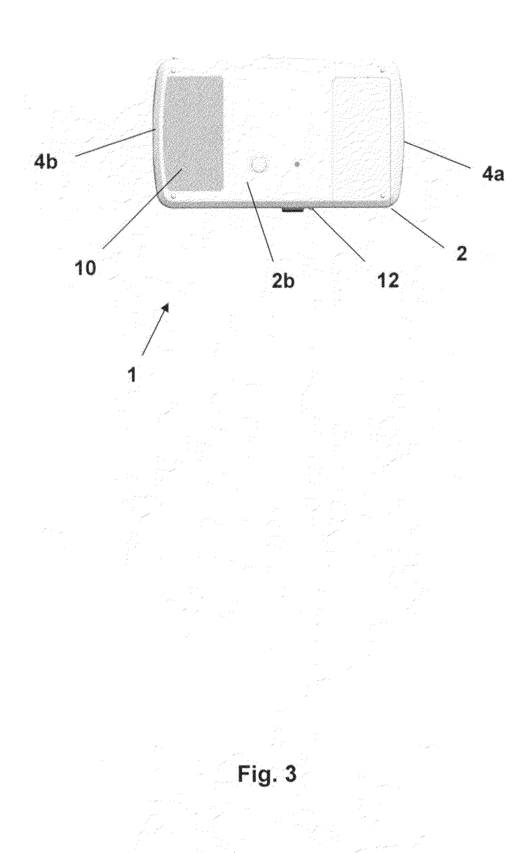

FIGS. 2 and 3 show a hand-held portable device (1) that is used in performing the method steps of the first embodiment. The device (1) includes a rectangular shaped housing (2), a processor (3) disposed in the housing (2) which is a SZ328 processor, a first and second input terminal (4a,4b) disposed on the housing, and a liquid crystal display (LCD) (5) mounted on the housing (2). The device also includes both a USB-compatible connector port (17) and a wireless communication interface based on Wi-Fi standard protocols (not shown) to facilitate communication with external devices if required.

The device (1) further includes a memory store (6a,6b) containing a program which is executable by the processor (3) in order to perform the method steps in accordance with the first embodiment of the present invention. That is, the program is adapted to perform, amongst other things, the following functions:
(a) detecting an ECG signal between the first and second input terminals (4a,4b);
(b) storing the ECG signal in a memory store (6a,6b);
(c) processing the ECG signal so as to identify at least one characteristic of the detected ECG signal;
(d) comparing the characteristic of the ECG signal against a predetermined abnormality criteria in order to determine if the ECG signal may be indicative of an abnormal functioning of the heart;
(e) outputting ECG and/or heart rate information to the LCD based on the processed ECG signal;
(f) communicating with external devices.

The memory store (6a,6b) of this embodiment includes 100 Kb on-chip eSRAM (6a), and, two 4 megabytes of flash memory (6b) which is provided in the form of a removable memory module. It should be noted that alternative types of memory devices and/or having larger capacity can be used instead depending upon the particular requirements of the application and the needs of the user. The memory store (6a,6b) is also used to store recordings of ECG signals that are detected by the processor (3) in use. At least about 20 recordings are able to be stored in the memory at any one time so that the user is provided with a historical log of past readings. Portions of the memory store (6a,6b) are able to be selectively erased by the user for instance, to provide space for more new recordings.

The processor (3) is operably connected with each of the first and second input terminals (4a,4b), the memory store (6a,6b) and the LCD (5).

The housing (2) includes an upper and a lower surface (2a,2b), and, left and right ends. The left and right ends of the housing (2) are defined from the point of view of a user when ordinarily reading the LCD (5). The LCD (5) is mounted on the upper surface (2a) of the housing (2).

The first and second input terminals (4a,4b) include electrically conductive metallic panels. The first and second input terminals (4a,4b) are mounted on the upper surface (2a) of the housing (2) on right end and left end of the housing (2) respectively.

The device (1) is powered by either two AA batteries (7a) or via a mains power supply (7b). The device further includes a user-control interface in the form of push-buttons which allows the user to selectively enable amongst other things:
(a) the activation/deactivation of power to the device (1);
(b) management of space in the memory store (6a,6b);
(c) transmission of recorded ECG signals to external devices; and
(d) modes of displaying information on the LCD (5).

The device (1) is adapted for use in processing both Lead-1 and Lead-2 type ECGs. The steps involved in generating a Lead-1 type ECG will be described first and will be followed thereafter by a description of how a Lead-2 type ECG is taken using the device.

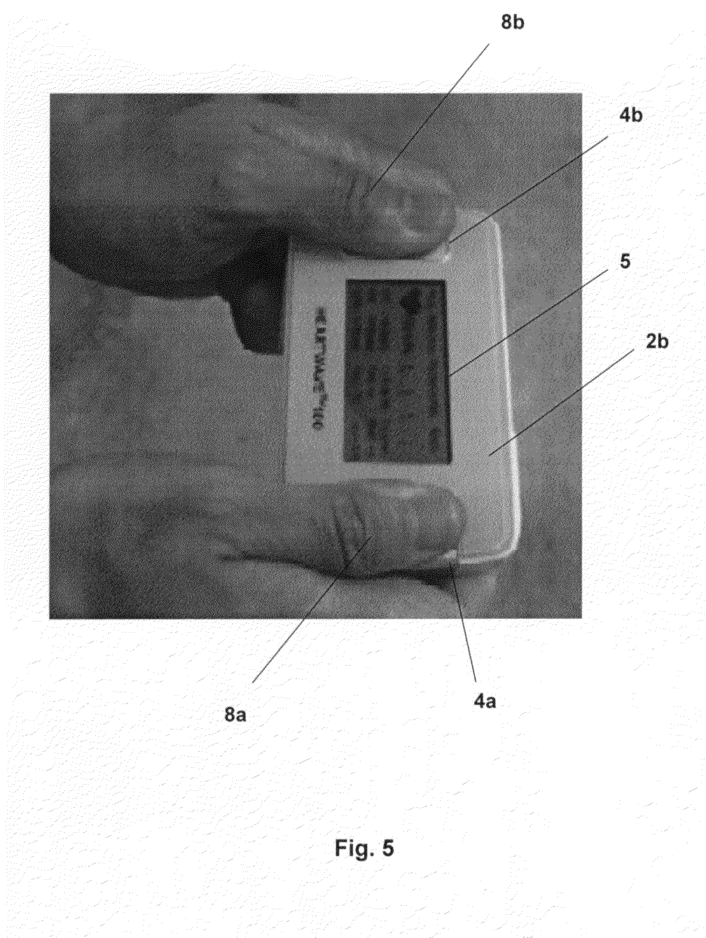

Referring now to FIG. 5 and the flow-chart of FIG. 1, in order to record a Lead-1 type ECG, a right thumb of the user (8a) is contacted with the right input terminal (4a) of the device (1), and, the user's left thumb (8b) is contacted with the left input terminal (4b) of the device (1) at the same time so as to form a closed electrical circuit with the device (1).

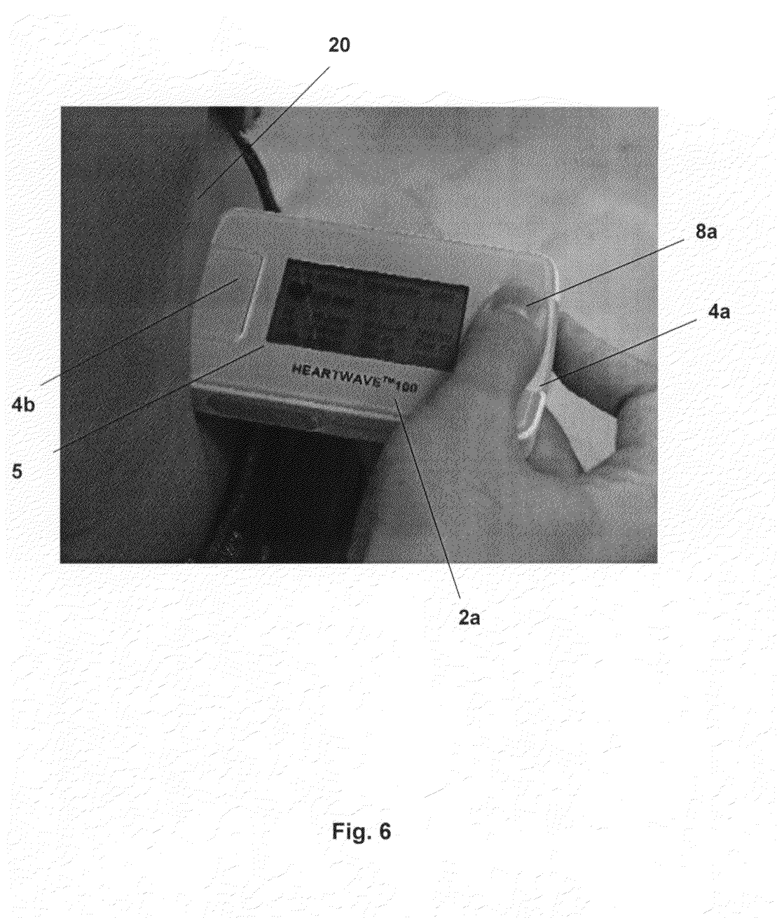

Alternatively, a Lead-2 type ECG can be recorded by contacting the user's right thumb (8a) with the first input terminal (4a), and, contacting a region of skin disposed on a lower-left section of the user's abdomen (20) with the second input terminal (4b), as shown in FIG. 6.

The step of contacting the device with the user's body in order to generate either of a Lead-1 or Lead-2 type ECG is represented by block (100) in FIG. 1.

Whether taking a Lead-1 or a Lead-2 type reading, the first and second input terminals (4a,4b) should be held in contact with the appropriate part of the user's body for a duration of at least approximately 20 seconds so that a sample of the ECG signal can be recorded. Accordingly, each ECG signal that is recorded is approximately 20 seconds in duration.

Referring now to the flow-chart of FIG. 1, the processor (3) is able to detect an ECG signal formed between the first and second input terminals (4a,4b) which is indicative of the health status of the user's heart. This step is represented by block (200) in FIG. 1A common-mode voltage signal associated with the detected ECG signal is reduced using a noise reduction module (9) which includes a common mode operational amplifier. The noise-reduction module (9) derives a common-mode voltage signal from between the first and second input terminals (4a,4b) during recording of the detected ECG signal, inverts the derived common-mode voltage signal, and drives the inverted common-mode voltage signal back into the user via a metal plate (10) which is placed in contact with the user's skin during recordal of the ECG signal. The metal plate is disposed on the lower surface (2b) of the device housing (2) as shown in FIG. 3.

The detected and noise-reduced ECG signal arising between the first and second input terminals (4a,4b) is recorded by the device and stored in the memory store (6a,6b) of the device. The step of effecting noise reduction and recordal of the detected ECG signal is represented in block 300 in FIG. 1.

Processing software stored in the memory (6a,6b) is adapted to process the recorded signal in order to analyse the signal. For instance, the software will measure a heart rate of the user by reference to the recorded ECG signal as well as identify at least one of the following characteristic of the ECG signal:
(a) an ST segment of the ECG signal;
(b) a PR segment of the ECG signal;
(c) a PR interval of the ECG signal;
(d) a QT interval of the ECG signal;
(e) a QRS complex of the ECG signal.

The step of analysing the recorded ECG signal is represented by block (400) in FIG. 1.

Thereafter, information is displayed on the LCD (5) by reference to the recorded ECG signal. The information (5a) includes at least one of, a graphic representation of the ECG signal (eg. voltage vs time), a summary of measured characteristics of the recorded ECG signal, and/or, a heart rate of the user. The step of displaying information to the LCD (5) is represented by block (500) in FIG. 1

The user is able to control which information is actually displayed to the LCD by operating a user-control (12) interface consisting of push-buttons (12a) disposed on the housing (2). FIG. 3 depicts an example of the format in which information is displayed to the LCD (5). For instance, user's heart rate and ECG can be outputted concurrently to the LCD (5), or, interchangeably.

The first embodiment also enables the user to identify any abnormalities in the behaviour of the recorded ECG signal which may be indicative of a heart condition. The detection of abnormalities is performed either by the device (1) itself, or, by an external server (13). The user can make a selection as to which option will be taken. This step is represented by block (600) in FIG. 1.

The user is also able to program the device to conduct either a manual or automatic diagnosis of the recorded ECG signal in order to determine if an abnormality is affecting the user's heart. The step of identifying if an abnormality exists, or has worsened, involves the identified characteristics of the user's recorded ECG signal being compared against a predetermined abnormality criteria wherein the criteria includes sets of parameters associated with ECG signals of hearts suffering from various heart abnormalities such as an arrhythmia (e.g. bradycardiac, tachycardiac, and/or premature ventricular contraction). By comparing the user's recorded signal characteristics with the sets of parameters, a determination is able to be made as to whether there is some risk that the user's heart may also be suffering from any one of the heart abnormalities. The actual comparison is able to be effected using any number of existing software applications and a person skilled in the art would be considered capable of selecting one such application accordingly. The step of the device (1) detecting an abnormality in the recorded signal is represented by block (700) of FIG. 1.

Typically, the predetermined abnormality criteria may include at least one parameter defining how the characteristic of the detected ECG signal should typically behave when the user's heart is functioning normally. For instance, the criteria may require that the ST segment be of a particular duration.

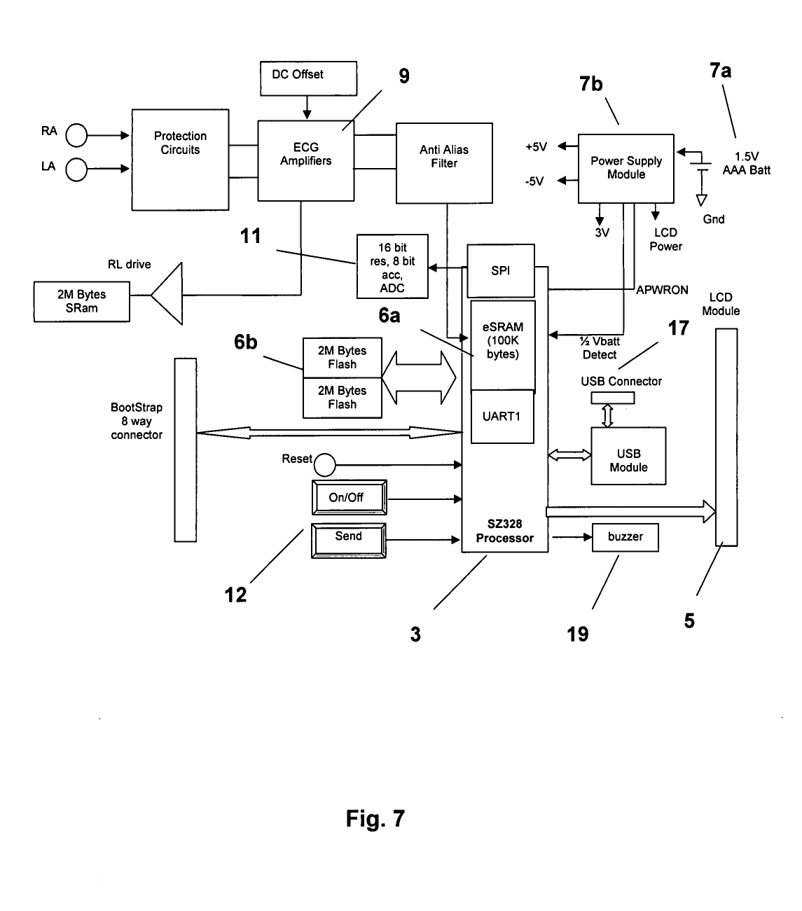

If an abnormality is detected, the processor (3) is programmed to automatically output an appropriate warning message to the LCD (5). The warning message will indicate the nature of the detected abnormality, as well as information concerning what steps to take in alleviating the abnormality before proper medical attention arrives. An audio alarm (19) is also provided as shown in the electrical schematic diagram of FIG. 7 which can be programmed by a user to activate when an abnormality is detected. This step is represented by block (800) in FIG. 1.

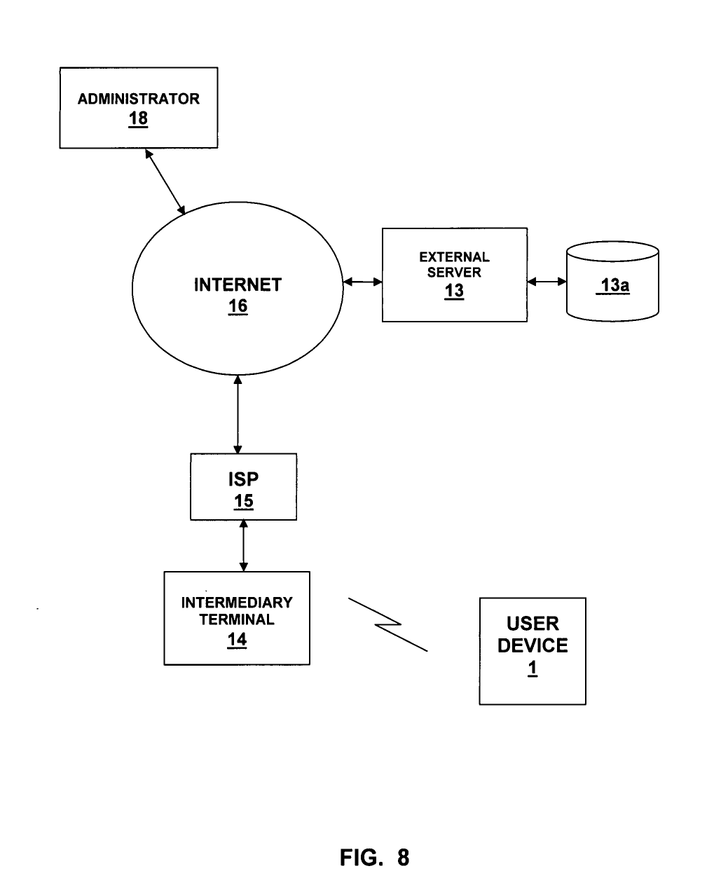

As mentioned above, the step of comparing the characteristic of the detected ECG signal against the predetermined abnormality criteria is able to be performed by the processor (3) of the device itself, or alternatively, by an external server (13) communicably connected to the device (1) via the Internet (16). A network diagram depicting the connection between the device (1) and the external server (13) is shown in FIG. 8. The control means disposed on the device (1) allows the user to select between whether the recorded ECG signal is to be analysed by the device (1) itself, or, by the external server (13). In the latter case, the device (1) will seek to first establish a connection with the external server (13) if none exists, whenever an analysis of the recording is sought by the user. This step is represented by block (900) in FIG. 1. When a connection with the external server (13) exists, then the device (1) will initiate a transfer of the recorded ECG signal to the external server (13) via the an ISP (15) gateway to the Internet (16).

The device (1) transmits the recordings to an intermediary terminal (14) via a Wi-Fi interface and the intermediary terminal (14) thereafter sends the recordings to the external server (13) to be processed. The step of the device (1) transmitting the recorded ECG signal to the external server (13) is represented by block (1000) in FIG. 1. The external server (13) includes a database (13a) so that it can store any recordings it receives from the user. The step of the external server (13) receiving and storing the recorded ECG signal is represented by block (1100) in FIG. 1.

The external server (13) includes processing software similar to that installed on the device (1) itself which enables it to analyse the ECG signal received form the user's device (1) in order to detect any abnormalities in heart function of the user. This step is represented by block (1200) in FIG. 1.

In the event that an abnormality is actually detected by the external server (13), the external server (13) will also automatically send a warning message to the user. The external server (13) will send the warning message to the user via at least one of the following messaging formats:
(a) e-mail;
(b) SMS;
(c) Facsimile;
(d) a telephone call.

The external server (13) is also programmed to communicate with the device via the Internet (16) if an abnormality is detected, and, the device (1) would be caused to output an appropriate warning message on the LCD (5) of the device (1). The step of the external server (13) sending a warning message to the user is represented by block (1300) in FIG. 1.

It would be appreciated by a person skilled in the art that in order for the external server (13) to be able to send a message to the user in the above messaging formats, the external server would generally have a record of the user's contact details—eg. e-mail address, mobile phone number, home telephone number, facsimile number and so on. These details would be supplied to the external server (13) by the user upon registering to use the services of the external server (13).

A Web site interface (not shown) is also provided to allow a user to review his/her information relating to recordings of ECG signals that he/she has sent to the external server (13) from the device (1) for processing. The user can interact with the Web site via a PC having suitable Web browser software installed. The Web site provides, amongst other things, at least the following information to each user:
(a) details of each recording such as the measured heart rate associated with the recording;
(b) details of various segments of each recorded ECG signal such as the ST interval, QRS complex and the like;
(c) details of the time at which each recording was made;
(d) details of any abnormality(s) that have been detected a recording;
(e) details of any advice provided by a medical practitioner that has been requested by the user to review a recording;

The Web site is also interactive in that the user is able to personalise the presentation of information on the Web site. This involves for instance, submitting one or more presentation preferences to the external server (13) which instructs the external server (13) as to how information should be presented. A standard online template is provided for the user to enter and submit such preferences.

The Web site is hosted on a Web server which in this case is the external server (13) itself. The Web site includes a password-access scheme in order to ensure that personal information of each user is maintained in a confidential manner. The Web site also provides a means of effecting payment electronically for any fees which are incurred by the user from use of the external server (13) and/or the Web site. Payment can be made by the user submitting credit card details via an on-line payment form which when submitted to the external server (13) for processing assists in automating the payment transaction electronically from the relevant credit card account.

The Web site also provides a user-interface by which the user is able to manage the uploading of a recording directly to the external server (13). For instance, if the recording of a particular ECG signal is stored on the device (1) and the device (1) is interfaced with the PC via a USB cable for instance, then the user may, via the Web site, initiates a file transfer of the recording from the device (1) to the external server (13) for processing as required.

The first embodiment can also be placed in continuous contact with the user's body in accordance with either of the Lead-1 and Lead-2 procedures so as to continuously monitor the health status of the user's heart. In this arrangement, the ECG signal is processed regularly by either the device (1) itself or the external server (13) in order to determine if a heart abnormality is present. This mode of operation may be particularly useful in situations where it is important to regularly monitor any deterioration in heart function of the user as early detection of the heart abnormality provides a head start in preventative and/or corrective intervention.

At least some of the method steps of the present invention are implemented by an administrator (18) which may for instance be a hospital administration unit. A fee is charged by the administrator (18) to the user based on the number of recordings that are uploaded to the external server (13), and/or, the number of messages which are sent to the user. Additional medical consultation fees are charged to the user if the user requests review of the recording by an actual medical practitioner.

Revenues may also be generated in return for providing third-party organizations with advertising space on the Web site. For instance, advertisers could include specialist medical practitioners such as cardiologists and the like, or suppliers of pharmaceutical products and so on. It is conceivable that advertising can also be intermittently sent to the user's device from the external server whenever a connection exists.

Other fee schemes can be employed such as charging the user a flat fee for an unlimited number of recordings to be uploaded and processed and report on in a given time period. The fee transactions can be settled using any suitable electronic payments systems known to the person skilled in the art.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described without departing from the scope of the invention. All such variations and modification which become apparent to persons skilled in the art, should be considered to fall within the spirit and scope of the invention as broadly hereinbefore described. It is to be understood that the invention includes all such variations and modifications. The invention also includes all of the steps and features, referred or indicated in the specification, individually or collectively, and any and all combinations of any two or more of said steps or features.

1. A method of recording information for use in generating a Lead-1 type ECG indicative of a health status of a user's heart, the method being operable using a device which includes:
a housing;
a processor disposed in the housing;
a first and a second input terminal disposed on the housing, wherein the first and second input terminals are operatively connected to the processor; and
a display operatively connected to the processor;
wherein the method includes the steps of:
(i) contacting a right and left thumb of the user with the first and second input terminals respectively; and
(ii) the processor detecting an ECG signal formed between the first and second input terminals, wherein the ECG signal is indicative of the health status of the user's heart.

2. A method of recording information for use in generating a Lead-2 type ECG indicative of a health status of a user's heart, the method being operable using a device which includes:
a housing;
a processor disposed in the housing;
a first and a second input terminal disposed on the housing, wherein the first and second input terminals are operatively connected with the processor; and
a display operatively connected to the processor;
wherein the method includes the steps of:
(i) contacting a user's right thumb with the first input terminal, and, contacting a region of skin on the user's body with the second input terminal; and
(ii) the processor detecting an ECG signal formed between the first and second input terminals, wherein the ECG signal is indicative of an operating status of the user's heart.

3. A method as claimed in claim 1 including the step of recording the detected ECG signal.

4. A method as claimed in claim 3 wherein the first and second input terminals include electrically conductive panels.

5. A method as claimed in claim 4 wherein the first and second input terminals are disposed on an upper surface of the housing.

6. A method as claimed in claim 4 wherein the first and second input terminals are disposed at opposing ends of the housing.

7. A method as claimed in claim 3 wherein the second input terminal includes an extendable and retractable lead.

8. A method as claimed in claim 3 wherein the display includes a liquid crystal display (LCD).

9. A method as claimed in claim 8 wherein the LCD is disposed on the upper surface of the housing.

10. A method as claimed in claim 3 wherein the device includes a memory store adapted for storing a recording of the detected ECG signal.

11. A method as claimed in claim 10 wherein the memory store includes at least one of
(a) eSRAM;
(b) RAM;
(c) Flash Memory;

12. A method as claimed in claim 10 wherein the memory store is adapted to store at least 20 recorded ECG signals.

13. A method as claimed in claim 3 wherein the device includes a communication interface to enable communication with an external computing device.

14. A method as claimed in claim 13 wherein the communication interface includes at least one of a wired or wireless interface.

15. A method as claimed in claim 14 wherein the wireless interface includes at least one of a Bluetooth-compatible interface, Wi-Fi compatible interface, or infra-red compatible interface.

16. A method as claimed in claim 14 wherein the wired interface includes a USB-compatible connector.

17. A method as claimed in claim 3 wherein contact is maintained between the user and the device in accordance with either step (i) of claim 1 and step (i) of claim 2 for a duration of at least about 20 seconds when recording the detected ECG signal.

18. A method as claimed in claim 3 wherein, in step (i) of claim 2, the region of skin of the user's body surface against which the second input terminal is contacted with includes a region of skin disposed on a lower-left section of the user's abdomen.

19. A method as claimed in claim 3 including the step of applying noise-reduction to the detected ECG signal so as to reduce a common-noise component associated with the ECG signal.

20. A method as claimed in claim 19 wherein the step of applying noise reduction to the ECG signal includes the use of a common-mode operational amplifier.

21. A method as claimed in claim 20 wherein the common mode operational amplifier derives a common-mode voltage signal from between the first and second input terminals, inverts the derived common-mode voltage signal, and drives the inverted common-mode voltage signal into the user's body.

22. A method as claimed in claim 21 wherein the inverted common-mode voltage signal is driven into the user's body via a metal plate disposed on the housing when the metal plate is contacted against the user's skin.

23. A method as claimed in claim 22 wherein the metal plate is disposed on a rear surface of the housing.

24. A method as claimed in claim 3 including the step of measuring at least one characteristic of the recorded ECG signal.

25. A method as claimed in claim 24 wherein the at least one characteristic of the recorded ECG signal includes at least one of:
(a) an ST segment of the ECG signal;
(b) a PR segment of the ECG signal;
(c) a PR interval of the ECG signal;
(d) a QT interval of the ECG signal;
(e) a QRS complex of the ECG signal;
(f) a heart rate indicated by the ECG signal.

26. A method as claimed in claim 24 including the step of outputting at least one of the characteristics of the ECG signal to the display.

27. A method as claimed in claim 24 including the step of identifying at least one measured characteristic of the recorded ECG signal which is indicative of a heart abnormality.

28. A method as claimed in claim 27 wherein the heart abnormality includes a heart arrhythmia.

29. A method as claimed in claim 27 including the step of comparing the at least one measured characteristic of the ECG signal against a predetermined abnormality criteria, wherein if the measured characteristic meets the predetermined abnormality criteria, an abnormality is detected.

30. A method as claimed in claim 29 including the step of outputting a message to the user when an abnormality is detected.

31. A method as claimed in claim 30 wherein the message includes at least one of:
(a) a text message outputted to the display;
(b) an audible alarm being activated;
(c) a light-emitter being activated.

32. A method as claimed in claim 31 wherein the text message includes an indication as to the nature of the abnormality detected.

33. A method as claimed in claim 31 wherein the text message includes information about treating the abnormality.

34. A method as claimed in claim 27 wherein the step of identifying at least one measured characteristic of the recorded ECG signal which is indicative of a heart abnormality is performed by an external server.

35. A method as claimed in claim 34 wherein the external server is communicably connected to the device via a communication link including at least one of:
(a) the Internet;
(b) an Intranet;
(c) a local area network;
(d) a wide area network.

36. A method as claimed in claim 34 wherein the external server includes a database for storing the recorded ECG signal sent from the user.

37. A method as claimed in claim 34 including the step of the external server sending a warning message to the user when an abnormality is detected.

38. A method as claimed in claim 37 wherein the warning message is sent in accordance with at least one of the following messaging formats:
(a) e-mail;
(b) SMS;
(c) facsimile;
(d) a telephone call; and/or
(e) a text entry posted on an Internet Web site.

39. A method as claimed in claim 34 wherein the recorded ECG signal that is uploaded to the external server from the device is accessible by a medical practitioner, and the user is provided with an option of requesting review of the ECG signal by the medical practitioner whereby, the medical practitioner, in response to the request, provides the user with advice concerning the health of the user's heart by reference to the review of the recorded ECG signal.

40. A method as claimed in claim 34 including the step of providing a Web site via which the user is able to interact with the external server.

41. A method as claimed in claim 34 wherein the external server and/or Web site are managed by an administrator.

42. A method as claimed in claim 40 including the step of the administrator charging the user a fee in exchange for having a recorded ECG signal uploaded to the external server.

43. A method as claimed in claim 41 including the step of the administrator charging the user a fee in exchange for a medical practitioner reviewing an uploaded ECG signal in response to a request received from the user.

44. A method as claimed in claim 41 including the step of the administrator charging the user a fee in proportion to a number of recorded ECG signals that are uploaded by the user to the external server and/or stored.

45. A method as claimed in claim 41 including the step of the administrator providing advertising space on the Web site in exchange for payment of a fee.

46. A method as claimed in claim 1 wherein the device includes a user-control means adapted for allowing the user to control at least one of the following functional aspects of the device:
(a) enabling/disabling power to the device;
(b) enabling/disabling transmission of recorded ECG signals to and/or from the device;
(c) selecting whether the processor of the device, or, the external server, will be used to detect any abnormalities in a recorded ECG signal;

47. A method as claimed in claim 1 including the step of integrating a blood pressure reader into the device.

48. A device adapted for use in performing the method steps in accordance with claim 1.

49. A computer server adapted for use in performing the method steps in accordance with claim 1.

50. A computer readable medium for storing a computer program which is executable to perform the method steps in accordance with claim 1.
